(19) United States
(12) Patent Application Publication
(10) Pub. No.: US 2009/0112186 A1
(43) Pub. Date: Apr. 30, 2009

(54) Catheter assembly with increased torsional stiffness

(76) Inventor: Mark L. Adams, Sandy, UT (US)

Correspondence Address: KRIEGSMAN & KRIEGSMAN, 30 TURNPIKE ROAD, SUITE 9, SOUTHBOROUGH, MA, 01772, US

(21) Appl. No.: 12/286,904

(22) Filed: Oct. 2, 2008

Related U.S. Application Data
(60) Provisional application No. 60/997,200, filed on Oct. 2, 2007.

(51) Int. Cl. A61M 25/00

(52) U.S. Cl. 604/524

(57) ABSTRACT

A catheter assembly having improved torsional stiffness. In one embodiment, the assembly includes a medical catheter and a torsional stiffener. The medical catheter may be a tubular member made of a soft, flexible, biocompatible material that may be shaped to include a first end, a second end, a side wall and a longitudinal bore. The first end, which may be implanted within a patient, may be shaped to include an internal bolster. The torsional stiffener, which may be coaxially encapsulated within the side wall of the catheter, may be a tubular member shaped to include a first end, a second end, a side wall and a longitudinal bore. One or more transverse openings may be provided in the side wall of the stiffener to endow the stiffener with a desired degree of lateral flexibility while still maintaining a desired degree of torsional stiffness. As a result, the stiffener augments the torsional stiffness of the catheter without detracting substantially from the flexibility of the catheter.

### CROSS-REFERENCE TO RELATED APPLICATIONS

The present application claims the benefit under 35 U.S.C. 119(e) of U.S. Provisional Patent Application Ser. No. 60/997,200, filed Oct. 2, 2007, the disclosure of which is incorporated herein by reference.

### BACKGROUND OF THE INVENTION

The present invention relates generally to catheters, such as, but not limited to, percutaneous catheters, and relates more particularly to a novel catheter assembly having increased torsional stiffness.

Medical catheters are currently used in a variety of different situations. For example, certain types of medical catheters, such as gastrostomy tubes, jejunostomy tubes and naso-gastric tubes, are often used to administer fluids to patients who are unable to take food and/or medications orally. The inability of these patients to take food and/or medications orally may be due to, for example, head or neck injuries, facial paralysis, esophageal strictures, dementia, a state of vegetation or other diseases or medical conditions. In addition to medical catheters of the type described above that are used to administer fluids to patients, there also exist other types of medical catheters that are used to drain fluids from a patient. An example of such a medical catheter is a Foley catheter, which is used to remove urine from a patient who cannot otherwise void the urine by normal means. Conditions requiring such drainage may include, but are not limited to, a swollen prostate, a narrowing of the urethra, post-surgical dysfunction and spinal cord damage. Still other types of medical catheters include PICC (peripherally inserted central catheter) lines, nephrostomy catheters, and other percutaneous catheters.

Traditionally, medical catheters are made of a low durometer, flexible material in order to minimize the abrasion of the patient's tissues following contact with the catheter. By minimizing such abrasion, the catheter is made to feel more comfortable to the patient. However, one problem that is commonly encountered in connection with medical catheters is that the catheters have a tendency, over time, to adhere to the tissues with which they are in contact. More specifically, in the case of gastrostomy and jejunostomy feeding tubes, such catheters have a tendency to come into contact with and to adhere to the abdominal and stomach tissues or abdominal and jejunal tissues, respectively, that are exposed in the open incision site through which the catheter is inserted. In the case of urinary drainage catheters, such catheters have a tendency to come into contact with and to adhere to the inner lining of the urinary tract. In the case of naso-gastric tubes, such catheters have a tendency to come into contact with and to adhere to nasal tissues, as well as the inner lining of the alimentary tract and the stomach. Although the reasons for adhesion are not entirely understood, it is believed that adhesion may be caused by a biofilm of bacteria on the outside surface of the catheter, said biofilm acting like a glue to adhere the catheter to the patient's tissue. It is also believed that adhesion may be caused by a lack of moisture between the tube and the patient's tissue. In any event, as can be appreciated, when the time comes to remove the catheter from the patient, such adhesion between the catheter and the patient's tissues often makes removal of the catheter very painful to the patient.

One approach to minimizing tissue adhesion to a feeding tube is disclosed in U.S. Patent Application Publication No. US 2004/0254545 A1, inventors Rider, II et al., which was published Dec. 16, 2004, and which is incorporated herein by reference. In this published application, there is disclosed a feeding tube that includes one or more surfaces having one or more anti-biofilm mechanisms. Such anti-biofilm mechanisms include impregnating or coating the tube with an antifungal agent.

Because the above approach uses chemical means, i.e., an antifungal agent, to reduce adhesion, the possibility exists that some patients may have an allergic reaction to the chemical means or may suffer other side effects related thereto. Moreover, there are certain complexities associated with the manufacture of a device that incorporates such chemical means, not to mention the difficulties in controlling the rate of elution of the chemical means from the tube. Furthermore, the incorporation of such an agent into the tube may adversely affect the structural integrity of the tube, i.e., the tube may become brittle, chemically unstable, etc.

In any event, a more common approach for minimizing the occurrence and severity of adhesion between a medical catheter and a patient is to move the catheter periodically, e.g., every 12 hours. It is typically recommended that such movement include both translational movement, i.e., moving the catheter back and forth a short distance within the patient, and rotational movement, i.e., rotating the catheter about its longitudinal axis within the patient. However, despite the fact that periodic movement of a catheter can substantially reduce the frequency and severity of adhesion problems, such movement is frequently neglected in many health-care settings. Moreover, even in those instances in which an effort is made to periodically move the catheter, such movement may be difficult to successfully achieve due to the physical properties of the catheter. This is because, as noted above, most catheters are made of a soft material. Although the use of a soft material results in a catheter that is comfortable to the patient, the use of such a soft material also frequently results in a catheter that lacks sufficient torsional stiffness to be easily rotated about its longitudinal axis. In fact, as a result of its low torsional stiffness, the catheter often simply becomes twisted when a rotational force is applied. One approach to this problem has been to use higher durometer, i.e., stiffer, materials to form the catheter since stiffer catheters are easier to rotate about their longitudinal axes. However, this approach has been unsatisfactory because catheters made from higher durometer materials are not as comfortable to patients.

### SUMMARY OF THE INVENTION

It is an object of the present invention to provide a novel catheter assembly.

It is another object of the present invention to provide a novel catheter assembly that possesses increased torsional stiffness.

Therefore, according to one aspect of the present invention, there is provided a catheter assembly comprising (a) a medical catheter; and (b) a torsional stiffener coupled to said medical catheter, said torsional stiffener substantially increasing the torsional stiffness of said medical catheter without substantially decreasing the flexibility of said medical catheter.

According to one embodiment, the catheter may be an elongated, tubular structure preferably made of a soft, flexible, biocompatible material. The catheter may be shaped to include a side wall terminating in a first end and in a second end, the side wall coaxially surrounding and defining a longitudinal bore preferably used to convey fluids, such as food and/or medications, to a patient in need thereof. The side wall may be generally cylindrical in shape but need not be. The first end of the side wall may be shaped to include an internal bolster for anchoring the catheter in a patient. The torsional stiffener may comprise a tubular stiffening member having high torsional stiffness. The tubular stiffening member may be coaxially encapsulated within the side wall of the catheter. The tubular stiffening member may be shaped to include a first end, a second end, a side wall and a longitudinal bore. One or more transverse openings may be provided in the side wall of the tubular stiffening member, the transverse openings being sized and located to endow the tubular stiffening member with a desired degree of lateral flexibility, i.e., bendability, while still maintaining a desired degree of torsional stiffness. In a preferred embodiment, the tubular stiffening member prevents kinking of the catheter. The tubular stiffening member may be a made of one or more suitable metals, such as a stainless steel or a shape memory alloy like nitinol (nickel-titanium alloy), or may be made of one or more suitable polymers, such as acrylonitrile butadiene styrene, polycarbonate, high-density polyethylene and the like.

According to another embodiment, the catheter comprises a coaxial combination of an inner tube and an outer tube, the inner tube and the outer tube being joined to one another, with a tubular stiffening member being sandwiched therebetween.

According to still another embodiment, the catheter comprises only an outer tube, with the tubular stiffening member being fixed to the inside of the outer tube.

According to still yet another embodiment, the tubular stiffening member may comprise a coil spring.

For purposes of the present specification and claims, various relational terms like “top,” “bottom,” “proximal” and “distal” are used to describe the present invention when said invention is positioned in or viewed from a given orientation. It is to be understood that, by altering the orientation of the invention, certain relational terms may need to be adjusted accordingly.

Additional objects, as well as features and advantages, of the present invention will be set forth in part in the description which follows, and in part will be obvious from the description or may be learned by practice of the invention. In the description, reference is made to the accompanying drawings which form a part thereof and in which is shown by way of illustration certain embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. In the drawings wherein like reference numerals represent like parts:

FIG. 1 is a fragmentary, perspective view, broken away in part, of a first embodiment of a catheter assembly constructed according to the teachings of the present invention;

FIG. 2 is a fragmentary, longitudinal, section view of the catheter assembly of FIG. 1;

FIG. 3 is an enlarged, perspective view of the torsional stiffener shown in FIG. 1;

FIG. 4 is a longitudinal, section view of a second embodiment of a catheter assembly constructed according to the teachings of the present invention; and

FIG. 5 is an enlarged side view of an alternate torsional stiffener that may be used, instead of the torsional stiffener of FIG. 3, in the catheter assembly of FIG. 1 or the catheter assembly of FIG. 4.

### DETAILED DESCRIPTION OF PREFERRED EMBODIMENTS

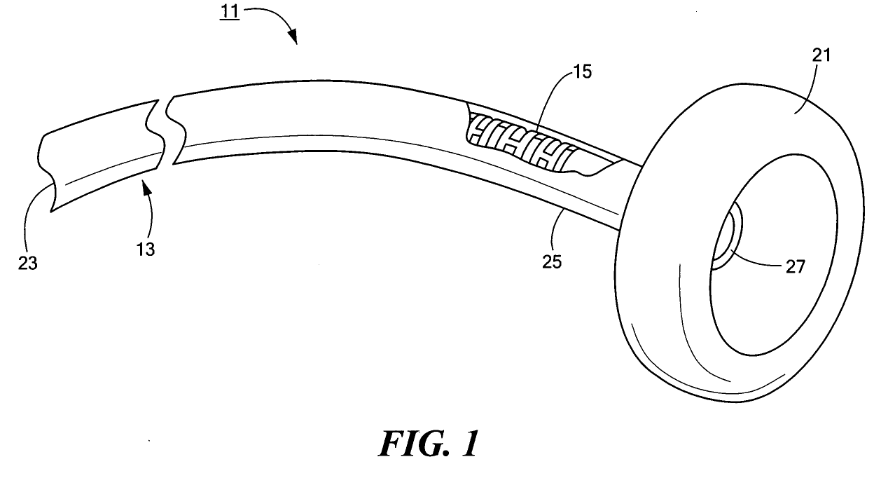

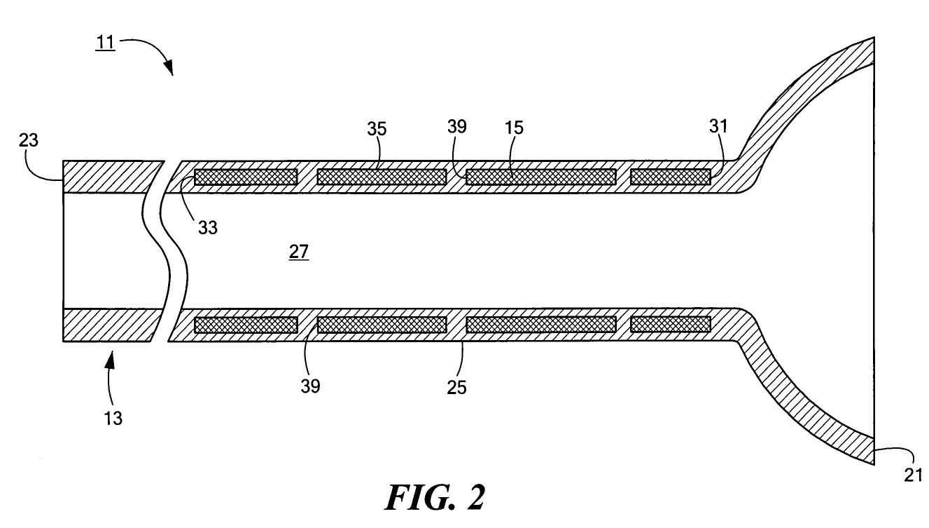

Referring now to FIGS. 1 and 2, there are shown fragmentary, perspective and fragmentary, longitudinal, section views, respectively, of a catheter assembly constructed according to the teachings of the present invention, said catheter assembly being represented generally by reference numeral 11.

Assembly 11 comprises a medical catheter 13 and a torsional stiffener 15.

Catheter 13, which may comprise a soft, flexible, biocompatible material, such as a low durometer polyurethane, nylon or silicone rubber, may be an elongated, cylindrical member shaped to include a first end 21, a second end 23, a side wall 25 and a longitudinal bore 27. (The flexibility of catheter 13 may also be affected by material thickness, by material fillers and/or by combined materials.) In the present embodiment, catheter 13 is a gastrostomy or jejunostomy feeding tube, and first end 21, which is intended to be implanted within a patient, is shaped to include an integrally-formed, dome-shaped, internal bolster. However, it should be understood that the catheter of the present invention is not limited to the specific structure described above. For instance, the internal bolster may be, for example, a balloon or a collapsible disk. Moreover, the catheter of the present invention need not be a gastrostomy or jejunostomy feeding tube, but instead, may be another type of percutaneous medical catheter or may be a Foley catheter, a naso-gastric tube or another type of catheter.

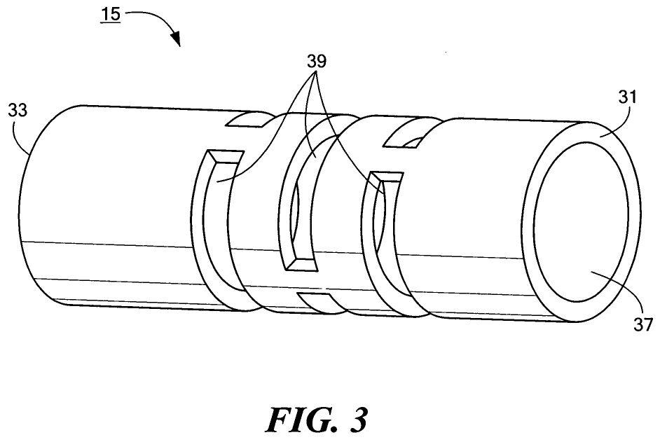

Torsional stiffener 15, which may be encapsulated within side wall 25 of catheter 13 and which may lie coaxial with catheter 13, is a generally tubular member having high torsional stiffness. Stiffener 15, which is illustrated separately in FIG. 3, may be shaped to include a first end 31, a second end 33, a side wall 35 and a longitudinal bore 37. One or more partial or complete transverse cuts, slots, grooves, gaps or openings 39 (hereinafter “openings”) may be provided in side wall 35, openings 39 being sized, shaped and located in side wall 35 in such a manner as to endow stiffener 15 with a desired degree of lateral flexibility, i.e., bendability, around its circumference while still maintaining a desired degree of torsional stiffness. (The flexibility of stiffener 15 may also be affected by material thickness, by material fillers and/or by combined materials.) Although openings 39 are shown in the present embodiment having a particular shape, size and location, openings 39 are not limited to the shape, size and location shown and may assume any of a number of alternate shapes, sizes and locations suitable to endow stiffener 15 with a desired stiffness and flexibility. In particular, the spacing between adjacent openings need not be uniform, and adjacent openings may vary in size, shape and depth from one another. Other variations in the openings may be similar to those disclosed, for example, in U.S. Patent Application Publication No. US 2004/0181174 A2, published Sep. 16, 2004, which is incorporated herein by reference. Stiffener 15 may be substantially uniform in stiffness over its length or may vary in stiffness over its length.

Stiffener 15 may be made of one or more suitable metals, such as a stainless steel or a shape memory alloy like nitinol (nickel-titanium alloy), or may be made of one or more suitable polymers, such as acrylonitrile butadiene styrene, polycarbonate, high-density polyethylene and the like. If made of metal, stiffener 15 may be formed, for example, by machining openings 39 in a unitary tubular member or by other metal-forming techniques, such as metal injection molding, casting, extruding, etc. (Additional information relating to techniques for forming openings 39 is disclosed in U.S. Pat. No. 5,690,120, inventors Jacobsen et al., which issued Nov. 25, 1997, and which is incorporated herein by reference.) If made of polymer, stiffener 15 may be made, for example, by various molding techniques. Preferably, stiffener 15 may be dimensioned lengthwise and may be positioned translationally relative to catheter 13 so that, once assembly 11 is implanted in a patient, end 33 of stiffener 15 will be externally accessible and end 31 will be positioned just in front of first end 21 or, at a minimum, will extend to a point beyond where tissue adhesion may occur.

It should be understood that, although assembly 11 is shown in the present embodiment having one torsional stiffener 15, the assembly of the present invention may include a plurality of torsional stiffeners, for example, arranged in a connected or disconnected series.

To form assembly 11, catheter 13 may be molded, e.g., by extrusion, over stiffener 15. Alternative techniques include dipping, dripping, heat-shrinking, etc.

Assembly 11 may be implanted in a patient, used in a patient, and withdrawn from a patient in essentially the same manner as are conventional catheters, the principal difference being that, due to the construction of assembly 11, catheter 13 may be more easily rotated about its longitudinal axis than are conventional catheters.

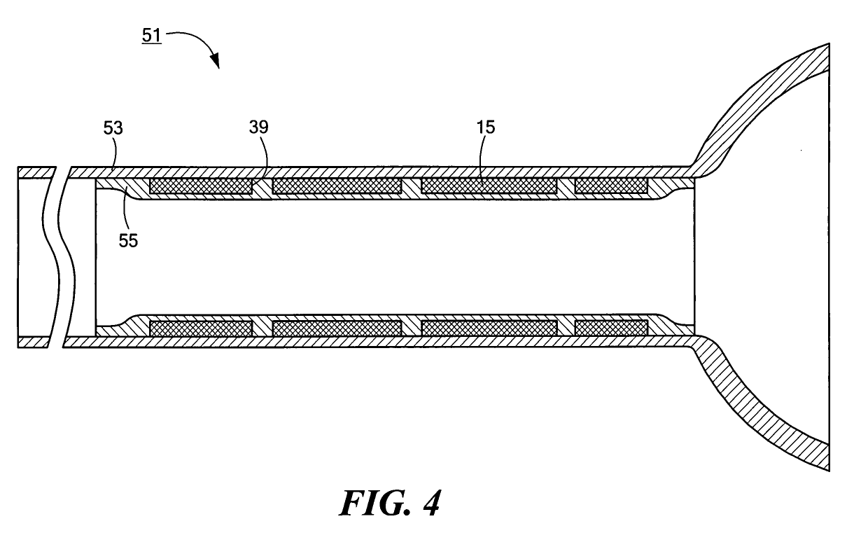

Referring now to FIG. 4, there is shown a longitudinal, section view of a second embodiment of a catheter assembly constructed according to the teachings of the present invention, said catheter assembly being represented generally by reference numeral 51.

Assembly 51 is similar in many respects to assembly 11, the principal difference between the two assemblies being that, whereas assembly 11 may include a catheter 13 within which stiffener 15 is encapsulated, assembly 51 may include an outer catheter 53 and an inner catheter 55 between which stiffener 15 is sandwiched. Outer catheter 53 and inner catheter 55 may be joined to one another by any one or more of a number of suitable techniques including welding, adhesive bonding, mechanical fastening, and the like.

In other embodiments, inner catheter 55 may be eliminated, and stiffener 15 may be fixedly or removably attached to the inside or outside of outer catheter 53.

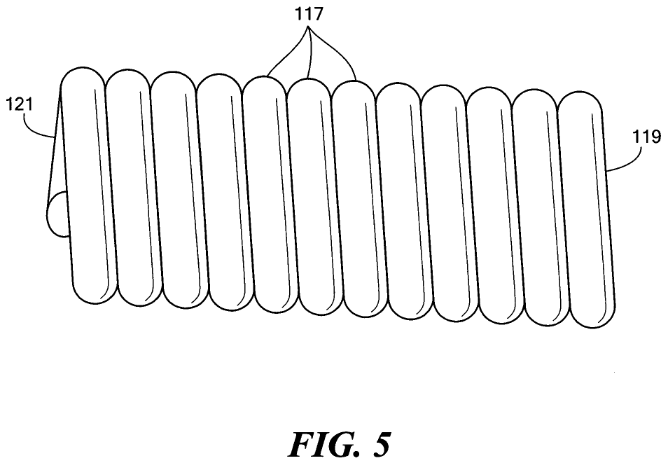

Referring now to FIG. 5, there is illustrated an enlarged side view of an alternate torsional stiffener that may be used, instead of stiffener 15, in any of the catheter assemblies described above, said alternate torsional stiffener being represented generally by reference numeral 115.

Stiffener 115 may comprise a coil spring comprising a plurality of closely spaced coils 117 terminating at a first end 119 and a second end 121. Stiffener 115 may be a unitary member and may be made of metal, polymers or other biocompatible materials including stainless steel or a shape memory material. Coils 117 may comprise a wire or thread at any useful cross-section including, but not limited to, round wire, flat wire and wires of various cross-sectional geometries, such as squares or “D”-shaped wire. Coils 117 may be spaced from one another at any suitable pitch.

Like stiffener 15, stiffener 115 serves to provide torsional strength to an associated catheter. At the same time, stiffener 115, itself, is sufficiently flexible so as not to substantially diminish the flexibility of its associated catheter.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A catheter assembly comprising:
(a) a medical catheter; and
(b) a torsional stiffener coupled to the medical catheter, the torsional stiffener substantially increasing the torsional stiffness of the medical catheter without substantially decreasing the flexibility of the medical catheter.

2. The catheter assembly as claimed in claim 1 wherein the medical catheter has a side wall and wherein the torsional stiffener is encapsulated within the side wall of the medical catheter.

3. The catheter assembly as claimed in claim 2 wherein the medical catheter comprises an inner tube and an outer tube, the inner tube and the outer tube being separate pieces joined to one another, with the torsional stiffener being sandwiched therebetween.

4. The catheter assembly as claimed in claim 2 wherein the medical catheter is a unitary structure.

5. The catheter assembly as claimed in claim 1 wherein the medical catheter is a feeding tube.

6. The catheter assembly as claimed in claim 5 wherein the medical catheter is a gastrostomy feeding tube.

7. The catheter assembly as claimed in claim 5 wherein the medical catheter is a jejunostomy feeding tube.

8. The catheter assembly as claimed in claim 1 wherein the medical catheter includes an internal bolster.

9. The catheter assembly as claimed in claim 1 wherein the catheter is made of a biocompatible material.

10. The catheter assembly as claimed in claim 9 wherein the biocompatible material is a low durometer material selected from the group consisting of polyurethane, nylon or silicone rubber.

11. The catheter assembly as claimed in claim 1 wherein the torsional stiffener comprises a coil spring.

12. The catheter assembly as claimed in claim 11 wherein the coil spring is made from a metal.

13. The catheter assembly as claimed in claim 11 wherein the coil spring is made from a polymer.

14. The catheter assembly as claimed in claim 1 wherein the torsional stiffener comprises a tubular structure.

15. The catheter assembly as claimed in claim 14 wherein the tubular structure is shaped to include a first end, a second end, a side wall and a longitudinal bore, the side wall of the tubular structure being provided with one or more transverse openings sized and positioned to increase lateral flexibility.

16. The catheter assembly as claimed in claim 15 wherein the tubular structure is made of one or more metals.

17. The catheter assembly as claimed in claim 16 wherein the tubular structure is made of a material selected from the group consisting of a stainless steel and a shape memory alloy.

18. The catheter assembly as claimed in claim 15 wherein the tubular structure is made of one or more polymers.

19. The catheter assembly as claimed in claim 18 wherein the tubular structure is made of a material selected from the group consisting of acrylonitrile butadiene styrene, polycarbonate and high-density polyethylene.
